(19) United States
(12) Patent Application Publication
(10) Pub. No.: US 2026/0114787 A1
(43) Pub. Date: Apr. 30, 2026

(54) Olfactory Diagnostic Compositions and Kits, and Methods of Making and Using the Same

(71) Applicant: UNIVERSITY OF KANSAS, LAWRENCE, KS (US)

(72) Inventors: JENNIFER VILLWOCK, SHAWNEE, KS (US); MICHAEL HAGEMAN, LAWRENCE, KS (US); THAIS MOTRIA SIELECKI-DZURDZ, KENNETT SQUARE, PA (US)

(21) Appl. No.: 19/003,647

(22) Filed: Dec. 27, 2024

Related U.S. Application Data
(60) Provisional application No. 63/616,078, filed on Dec. 29, 2023.

(51) Int. Cl. A61B 5/00

(52) U.S. Cl. CPC A61B 5/4011; A61B 5/4064; A61B 5/4088

(57) ABSTRACT

The present invention is directed to olfactory diagnostic testing compositions and kits for detecting, screening and/or monitoring the progression of specific disease states or other conditions in a subject. In one aspect, the invention is directed to a method and testing kit using a pathology-specific panel of fragrances to detect, screen and/or monitor a specific disease state or condition. In another aspect, the invention is directed to a method of making diagnostic compositions having a specific complex fragrance such as an essential oil fragrance and diagnostic testing kits including such diagnostic compositions.

### CROSS-REFERENCE TO RELATED APPLICATIONS

This application is based on and claims priority to U.S. Provisional Application Ser. No. 63/616,078 filed on Dec. 29, 2023, which is incorporated herein by reference in its entirety.

### STATEMENT REGARDING FEDERALLY SPONSORED RESEARCH OR DEVELOPMENT

Not applicable.

### BACKGROUND OF THE INVENTION

The present invention is generally directed to the field of diagnostic testing based on olfactory dysfunction. More specifically, the invention is directed to compositions, kits and methods for use in the early detection, screening and/or monitoring of neurologic, cognitive and other disease states or conditions exhibiting olfactory deficiency, dysfunction, or differences as compared to healthy subjects.

### BRIEF SUMMARY OF THE INVENTION

The present invention is directed to olfactory diagnostic compositions and kits, and methods of making and using olfactory diagnostic compositions and kits, for the early detection, screening and/or monitoring of neurologic, cognitive and other disease states or conditions exhibiting olfactory deficiency, dysfunction, or differences as compared to healthy subjects. For example, the compositions, kits and methods of the present invention may be used in the detection or monitoring of neurocognitive disorders in humans such as Alzheimer's disease (“AD”), multiple sclerosis (“MS”), and traumatic brain injury (“TBI”).

In one aspect, the invention is directed to a method of detecting, screening and/or monitoring a specific disease state or other condition using an olfactory phenotype developed for the specific disease state or other condition. A pathology-specific panel of fragrances comprising a plurality of fragrances (e.g. lavender, lemon, clove, garlic), each of which may be provided in a plurality of concentrations (e.g. 1X, 2X, 4X) is selected based on the olfactory phenotype. The subject's ability to detect and identify the fragrances within the panel at the various concentrations is then tested. The subject's ability to detect and identify the fragrances within the panel (including the various concentrations of the fragrances) is then compared to the olfactory phenotype.

In one embodiment, the olfactory phenotype is developed by conducting olfactory phenotype testing on at least two cohorts of subjects: a first cohort of subjects suffering from the specific disease state or other condition and a second cohort that is not suffering from the specific disease state or other condition (the “normal” cohort). The olfactory phenotype testing includes prompting the subject to smell a fragrance from a phenotype testing panel of fragrances (preferably by providing the subject an aroma or nasal inhaler device dosed with the fragrance); prompting the subject to confirm whether an aroma could be detected, wherein if the answer is yes, further prompting the subject to identify the aroma from among a list of choices; if the answer is no, optionally repeating the prompting steps with a higher dose of the fragrance until the subject answers yes or reaches a maximum dose; and repeating the prompting steps with each fragrance in the panel of fragrances. The method additionally includes recording the responses of the subjects to the detection and identification prompts for each fragrance at the various concentrations, preferably in a computer database that may include additional data relating to the subjects such as age, gender, ethnicity, cultural or geographical background, and the results of other testing. It is noteworthy that cultural background can impact the results of olfactory testing based on familiarity of the subjects with different types of fragrances. For testing subjects from different cultural or geographical backgrounds, it is important to ensure that the phenotype testing panel of fragrances includes culturally or geographically appropriate fragrances for the cohorts being tested and that differences in cultural or geographical background are documented for purposes of analyzing the results of the testing.

In some embodiments, each of the fragrances in the phenotype testing panel of fragrances are complex fragrances such as essential oil fragrances. The complex fragrances are made from compositions that include more than one type of odorant. In, some embodiments, each of the fragrances in the phenotype testing panel of fragrances is a “synthetic” composition formulated to be recognized as an essential oil fragrance or other known complex fragrance. In this embodiment, the synthetic fragrance composition consists of a limited set of odorants in prescribed amounts. In some embodiments, the synthetic fragrance composition comprises a “base composition” consisting of the limited set of odorants in prescribed amounts and a “carrier” such as an oil. The concentration or dosage amounts of the synthetic fragrance composition can be varied by varying the amount of the base composition in the carrier. In some embodiments, the base composition consists of two to five different types of odorants, in some embodiments at least three different types of odorants. Using a formulated synthetic fragrance composition of known composition as opposed to a natural essential oil ensures uniformity and consistency in the results of the testing.

The method further includes comparing the responses of the first cohort to the responses of the second cohort to identify any unique patterns of responses by the first cohort. The collection of unique patterns of responses for the first cohort is the olfactory phenotype for that specific disease-state or condition. As discussed above, the olfactory phenotype may vary based on the cultural or geographical backgrounds of the subjects.

In one embodiment, the panel of pathology-specific fragrances selected for use in detecting, screening or monitoring a specific disease-state or condition is a grouping or set of fragrances for which the responses of the first cohort as to one or more concentrations of the fragrances create a unique pattern of responses that is different from the pattern created from the responses of the second cohort to the same grouping or set of fragrances and concentrations. In one embodiment, the panel of pathology-specific fragrances selected is a grouping or set of fragrances for which the responses of the first cohort as to one or more concentrations of each fragrance varies more significantly from the responses of the second cohort than the responses vary for other fragrances. As discussed above, the panel of pathology-specific fragrances selected for use in detecting, screening or monitoring a specific disease-state or condition may be different for subjects of different cultural or geographic backgrounds.

In another aspect, the invention is directed to an olfactory diagnostic testing kit and method for detecting, screening and/or monitoring a specific disease state or other condition using a pathology-specific panel of fragrances that is selective for the specific disease state or other condition. A pathology-specific panel of fragrances that is selective for a specific disease state or other condition is a panel of fragrances for which the olfactory ability to detect and identify the collection of fragrances in the panel at certain concentrations or doses (in other words, the pattern of detection and identification for the fragrances at various concentrations) is significantly different for subjects that have the specific disease state or other condition than subjects without the specific disease state or condition. In some embodiments, the pathology-specific panel of fragrances may be based upon an olfactory phenotype developed via the olfactory phenotype testing discussed above. In some embodiments, the diagnostic kit includes a pathology-specific panel of at least four different fragrances (some or all of which are included at different concentrations), in some embodiments the diagnostic kit includes a pathology-specific panel of four to ten fragrances (some or all of which are included at different concentrations), and in some embodiments the diagnostic kit includes a pathology-specific panel of four to six fragrances (some or all of which are included at different concentrations).

In some embodiments, the diagnostic kit includes at least one control fragrance and in some embodiments includes one to three control fragrances that are not selective for the specific disease state or other condition being detected, screened or monitored by the kit. In some embodiments, the fragrances within the pathology-specific panel are complex fragrances such as essential oil fragrances. The complex fragrances are made from compositions that include more than one type of odorant (e.g. single aromatic compound or molecule), in some embodiments at least two, in some embodiments two to ten different types of odorants, in some embodiments at least three different types of odorants and no more than eight, and in some embodiments three to five odorants. In some embodiments, the complex fragrance compositions used in the diagnostic kit comprise synthetic compositions formulated to be recognized as an essential oil fragrance or other known complex fragrance. In this embodiment, the synthetic fragrance composition consists of a limited set of odorants (e.g. a finite set of different single aromatic compounds or molecules) combined together in prescribed amounts. The synthetic fragrance composition may be produced by combining the odorants or aromatic compounds or molecules in the prescribed amounts, optionally within a carrier.

In one embodiment, an olfactory diagnostic kit and method for detecting, screening and/or monitoring AD includes a pathology specific panel of fragrances selected from the group consisting of lavender, spearmint, licorice, garlic, coffee, lemon, clove and combinations thereof. In some embodiments, an olfactory diagnostic kit and method for detecting, screening and/or monitoring AD comprises a panel of lavender, lemon, clove and garlic fragrances. In some embodiments, the panel of fragrances consists of lavender, lemon, clove and garlic fragrances.

In another embodiment, an olfactory diagnostic kit and method for detecting, screening and/or monitoring TBI includes a pathology specific panel of fragrances selected from the group consisting of licorice, garlic, lavender, coffee and combinations thereof. In some embodiments, an olfactory diagnostic kit and method for detecting, screening and/or monitoring traumatic brain injury comprises a panel of licorice, garlic, lavender and coffee. In some embodiments, the panel of fragrances consists of panel of licorice, garlic, lavender and coffee. In some embodiments, an olfactory diagnostic kit and method for detecting, screening and/or monitoring traumatic brain injury also includes control fragrances selected from the group consisting of rosemary, ginger, and combinations thereof. In some embodiments, an olfactory diagnostic kit and method for detecting, screening and/or monitoring traumatic brain injury comprises rosemary and ginger control fragrances. In some embodiments, the control fragrances consists of rosemary and ginger.

In some embodiments, the kit comprises a plurality of aroma or nasal inhalers, each dosed with a diagnostic fragrance from the pathology-specific panel of diagnostic fragrances and an answer key. In some embodiments, the answer key first prompts a subject to use the inhaler and confirm whether an aroma could be detected, wherein if the answer is yes, then the answer key prompts for identification of the aroma among a list of choices, wherein if the answer is no, the subject can try another inhaler with a higher dose of the diagnostic fragrance until either the subject reaches a maximum dose or is able to answer in the affirmative. By first asking the subject to confirm whether they can smell the aroma, the kit can distinguish between misidentification and lack of detection.

In some embodiments, the kit comprises aroma or nasal inhalers with a series of doses for each fragrance, such as double strength, quadruple strength, and octuple strength. In some embodiments if the subject is unable to detect odorant at the octuple strength concentration, they will be coded as completely anosmic. In some embodiments, if the subject indicates that they do detect the odorant, but are unable to correctly identify it at least twice, then the dosage can be increased until they are able to correctly identify it.

The answer key can take any suitable form, depending on the environment. In some cases, the answer key is a computer interface. In some cases, the answer key is a document, chart, card, or book. In some cases, the answer key is designed to be filled out by a medical professional recording the subject's responses. In other cases, the answer key is designed to be followed and filled out by the subject. In these embodiments, the answer key can include an additional prompt to make sure the subject selects the correct inhaler.

In another aspect, the invention is directed to a method of making formulated “synthetic” diagnostic fragrance compositions that can be used as diagnostic fragrances for detecting, screening and/or monitoring olfactory dysfunction and to diagnostic kits including such fragrance compositions. In some aspects, the fragrance compositions can be used in a pathology-specific panel of diagnostic fragrances for detecting, screening and/or monitoring a specific disease state or other condition as discussed above. The method comprises selecting a complex fragrance such as an essential oil fragrance that is desirable for use as a diagnostic fragrance; identifying the primary odorants (e.g. in some embodiments 3 to 5 different single aromatic compounds or molecules) contained within naturally occurring and/or commercially available compositions (the “reference compositions”) having the complex fragrance; characterizing the primary odorants to determine their chemical structure and determining the respective concentration of each primary odorant in the reference compositions; and blending the primary odorants together in amounts corresponding to their relative amounts in the reference compositions in a carrier oil.

In another aspect, the invention is directed to synthetic diagnostic compositions for lavender, clove, spearmint, lemon, peppermint, rosemary, eucalyptus, cinnamon and licorice fragrances.

Additional aspects of the invention, together with the advantages and novel features applicable thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 is a flow diagram of a method for developing an olfactory phenotype and selecting a pathology-specific panel of fragrances for use in the present invention.

FIG. 2 depicts the AROMA™ testing scores of a normal cohort and a cohort having confirmed traumatic brain injury (“TBI”).

FIG. 3 is an embodiment of an aroma or nasal inhaler that may be used with the methods and kits of the present invention.

FIG. 4 illustrates an example of a computing device that be used with the methods and kits of the present invention.

FIG. 5 is a comparison of selected peaks from a gas chromatography analysis of six different commercially available natural essential oils.

### DETAILED DESCRIPTION OF EXEMPLARY EMBODIMENTS

### Disease Specific Olfactory Phenotypes

An exemplary embodiment of the invention is directed to a diagnostic method and testing kit for detecting, screening and/or monitoring neurologic, cognitive and other disease states or conditions in a human subject exhibiting olfactory deficiency, dysfunction, or differences as compared to healthy subjects using an olfactory phenotype developed for the specific disease state or other condition. For example, in some embodiments, the specific disease state or other condition to be detected, screened or monitored may be Alzheimer's disease (“AD”), multiple sclerosis (“MS”), dementia, Parkinson's disease, autism spectrum disorders, epilepsy, stroke, depression or traumatic brain injury (‘TBI”), as well as genetic brain diseases/disorders including but not limited to leukodystrophies and Wilson's. A pathology-specific panel of fragrances is selected based on the olfactory phenotype, and the subject's ability to detect and identify fragrances from the pathology-specific panel of fragrances is tested. The subject's ability to detect and identify the fragrances within the panel (including the various concentrations of the fragrances) is then compared to the olfactory phenotype.

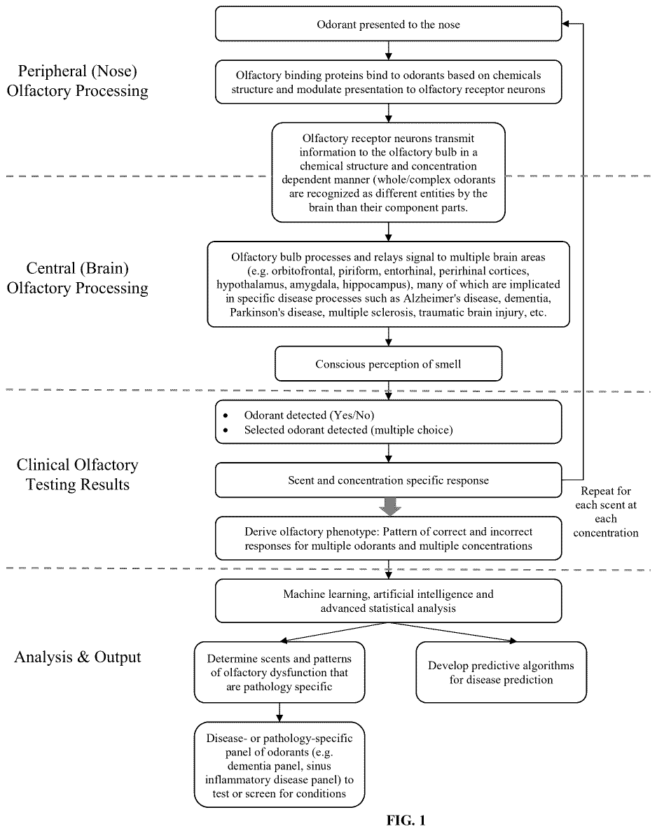

A method for developing an olfactory phenotype and selecting a pathology-specific panel of fragrances in accordance with an exemplary embodiment of the present invention is showing in FIG. 1. The olfactory phenotype is developed by conducting olfactory phenotype testing on at least two cohorts of subjects, a first cohort of subjects suffering from the specific disease state or other condition and a second cohort that is not suffering from the specific disease state or other condition. The olfactory phenotype testing includes prompting the subject to smell a fragrance or odorant from a phenotype testing panel of fragrances (preferably by providing the subject a nasal inhaler device dosed with the fragrance). The phenotype testing panel of fragrances may include a wide variety of different fragrances for testing at various concentrations. Preferably the fragrances included within the phenotype testing panel are complex odorants having a plurality of different odorants, preferably at least 3 odorants. In some embodiments, the fragrances used in the phenotype testing panel may be natural essential oils and in other embodiments the fragrances may be formulated or synthetic compositions made from a limited set of odorants in prescribed ratios that together are recognized as a specific essential oil fragrance.

Examples of known essential oil fragrances include Agar oil, Ajwain oil, Angelica root oil, Anise oil, Asafoetida oil, Balsam of Peru, Basil oil, Bay oil, Bergamot oil, Birch oil, Black pepper oil, Buchu oil, Camphor oil, Cannabis flower essential oil, Calamodin oil, Caraway seed oil, Cardamom seed oil, Carrot seed oil, Cedar oil, Chamomile oil, Calamus oil, Cinnamon oil, Citron oil, Citronella oil, Clary Sage oil, Coconut oil, Clove oil, Coffee oil, Coriander oil, Costmary oil, Costus root oil, Cranberry seed oil, Cubeb oil, Cumin seed oil, Cypress oil, Cypriol oil, Curry leaf oil, Davana oil, Dill oil, Elecampane oil, Elemi oil, Eucalyptus oil, Fennel seed oil, Fenugreek oil, Fir oil, Frankincense oil, Galangal oil, Galbanum oil, Garlic oil, Geranium oil, Ginger oil, Goldenrod oil, Grapefruit oil, Henna oil, Helichrysum oil, Hickory nut oil, Horseradish oil, Hyssop, Idaho-grown Tansy, Jasmine oil, Juniper berry oil, Laurus nobilis, Lavender oil, Ledum, Lemon oil, Lemongrass, Lime, Litsea cubeba oil, Linalool, Mandarin, Marjoram, Melissa oil, Mentha arvensis oil, Moringa oil, Mountain Savory, Mugwort oil, Mustard oil, Myrrh oil, Myrtle, Neem oil, Nutmeg oil, Orange oil, Oregano oil, Orris oil, Palo Santo, Parsley oil, Patchouli oil, Perilla essential oil, Pennyroyal oil, Peppermint oil, Petitgrain, Pine oil, Ravensara, Red Cedar, Roman Chamomile, Rose oil, Rosehip oil, Rosemary oil, Rosewood oil, Sage oil, Sandalwood oil, Sassafras oil, Savory oil, Schisandra oil, Spearmint oil, Spikenard, Spruce oil, Star anise oil, Tangerine, Tarragon oil, Tea tree oil, Thyme oil, Tsuga, Turmeric, Valerian, Warionia, Vetiver oil, Western red cedar, Wintergreen, Yarrow oil, Ylang-ylang, and Zedoary.

Also, the fragrances included within the phenotype testing panel should be culturally and geographically appropriate for the individual subjects who will ultimately be the subjects of the diagnostic screening or monitoring. Exposure to odors varies by culture and can impact performance on olfactory tests. For example, Chinese subjects perform 15% better on a culturally appropriate version of the UPSIT versus the standard version. For testing subjects from different cultural or geographical backgrounds, it is therefore important to ensure that the phenotype testing panel of fragrances includes culturally or geographically appropriate fragrances for the cohorts being tested and that differences in cultural or geographical background are documented for purposes of analyzing the results of the testing.

In one embodiment, the phenotype testing panel of fragrances is based on the Affordable, Rapid, Olfactory Measurement Array (AROMA™) which includes 14 different essential oil scents or fragrances in 4 concentrations (1×, 2×, 4×, 8×): garlic, clove, spearmint, licorice, lavender, coffee, peppermint, rosemary, eucalyptus, rose, orange, lemon and ginger. The 14 scents at 4 concentrations consist of a full battery of 56 inhalant sticks. However, not every individual is presented with every stick. The AROMA™ protocol typically requires that all scents are originally presented at the 2× concentration and that all scents at a particular concentration are completed before moving to the next round of testing at a different concentration. The sequence of odors for each concentration is randomized prior to presentation. A correct response requires both correct detection of an odor (scent detection is measured as “percent detected”) and correctly selecting the present odor among four multiple choices (scent identification is measured as “percent correct”). Failure to respond correctly results in being presented with the next higher concentration in random order during the next round of testing. Correct response at the 2× concentration results in the individual being presented with the 1× concentration and assumes correct responses at the 4× and 8× concentrations. As such, the maximum number of inhalant sticks presented to an individual is 42. In addition to the scores for individual scents at each concentration, a composite score for each scent is calculated. The maximum composite score is 100. AROMA™ is preferably administered in a proctored setting for purposes of developing an olfactory phenotype to ensure uniform methods in a research setting.

As noted in FIG. 1, an odorant (fragrance at a specific concentration) within the panel is presented to the nose of the subject. Olfactory binding proteins bind to the odorants based on the chemical structure of the odorants and modulate presentation of the odorant to olfactory receptor neurons. Olfactory receptor neurons transmit the information to the olfactory bulb in a chemical structure and concentration dependent manner. Whole/complex odorants are recognized as different entities by the brain than their component parts. The olfactory bulb processes and relays signal to multiple brain areas (e.g. orbitofrontal, piriform, entorhinal, perirhinal cortices, hypothalamus, amygdala, hippocampus), many of which are implicated in specific disease processes such as Alzheimer's disease, dementia, Parkinson's disease, multiple sclerosis, traumatic brain injury, etc. Using the AROMA™ testing protocol, the subject is prompted to confirm whether an aroma could be detected. If the answer is yes, the subject is prompted to identify the aroma from among a list of choices. If the answer is no, the presentation of the odorant at a higher concentration is repeated until the subject answers yes or reaches a maximum dose. The above steps are repeated for each fragrance at each concentration in the panel.

The responses of the subjects to the detection and identification prompts for each odorant are recorded and analyzed to derive an olfactory phenotype for each cohort, namely the pattern of correct and incorrect responses for each of the odorants at the various concentrations. The olfactory phenotype for the cohort suffering from the specific disease state or condition can be used as a biomarker for the disease state or condition that is cost-effective and deployable at the primary care and population health level.

The responses to the detection and identification prompts are preferably recorded in a computer database along with other data relating to the subjects such as age, gender, ethnicity, cultural or geographical background, and the results of other testing such as biological or clinical testing. All of this data can then be analyzed using processes and technologies known in the art including machine learning, artificial intelligence and advanced statistical analysis to determine the scents and patterns of olfactory dysfunction that are pathology specific. In other words, the data can be analyzed to determine those responses and patterns of responses of the cohort with the disease state or condition that are unique or significantly different from the responses and patterns of responses healthy cohort. Those unique responses or patterns of responses can be used to select the pathology-specific panel of scents or fragrances (and concentrations thereof) to include in a testing kit to accurately detect or predict the specific disease state or condition. Furthermore, machine learning or other analytical processes or technologies may be used to develop predictive algorithms that will assess a subject's responses to testing with the pathology-specific panel of fragrances for accurate disease prediction.

The inventors propose that resultant ML algorithms from this data analysis may be deployed via smart-phone applications or other technology-driven options to allow real-time use by clinicians to aid in diagnostic, therapeutic, and care planning decisions. It is believed that the association of high-dimensional olfactory data with biological and clinical disease markers can be determined via traditional statistics and machine learning (ML). In addition, the ML algorithms may ultimately be leveraged to classify the severity of disease such as TBI severity.

The testing used for developing an olfactory phenotype and the testing used to detect, screen and/or monitor a specific disease state or other condition may be scored electronically and in real-time via automated systems. For example, the data may be stored via a web-based system such as REDCap. This electronic interface is ideal for eventual deployment of resultant ML algorithms for point-of-care use in clinical, research, and community settings.

### TBI Testing and Kit

In one embodiment, the inventors propose to use olfactory dysfunction (OD) data obtained from AROMA™ testing and clinical data in combination with machine learning (ML) algorithms to define olfactory phenotypes that are reliable noninvasive biomarkers of TBI diagnosis and severity. Non-penetrating TBI the injury is the result of an external force which is strong enough to move the brain in the skull. TBI is characterized by physical symptoms (headache, seizures, blurred vision, nausea), cognitive/behavioral changes (confusion, irritability, decreased level of consciousness), as well as perception (light headedness, hearing issues, sensitivity to light and sound). Early diagnosis of all levels (mild, moderate and severe) of TBI is critical as early treatment has been shown to result in improved outcomes. Currently TBI is diagnosed based on knowledge of the injury, patient symptomology, and brain imaging, a time consuming process associated with higher costs. With so many individuals effected by TBI, an easy to use, in field, cost-effective diagnostic would be critical in helping these individuals initiate earlier treatment.

OD occurs in TBI and is correlated with injury severity. OD occurs in the preclinical stages of TBI due to the extensive neuroanatomic overlap of cognition and olfaction. It can exacerbate other neuropsychiatric symptoms and decrease quality of life. OD in this setting can be due to traumatic shearing of nerves at the olfactory cleft as well as damage to central olfactory processing structures and pathways during blunt head trauma. Commercially available tests of OD exist, but have not been effectively leveraged for TBI screening, diagnosis and clinical monitoring. Many of these prior tests use single, or simple mixtures, of chemicals. “Real world” olfaction requires processing of complex blends of odorant molecules because piriform cortex processing is dependent on odorant mixture composition. Many of the commercially available tests also do not assess both detection and identification and most do not provide data on scent- and concentration-specific performance, which the inventors believe is important for defining olfactory phenotypes predictive of disease. It is believed that complex odors are better suited for OD-testing for TBI (as well as for other disease-states and conditions) as they also may be more related to important functional metrics such as cognitive, physical and perception performance, and longitudinal performance.

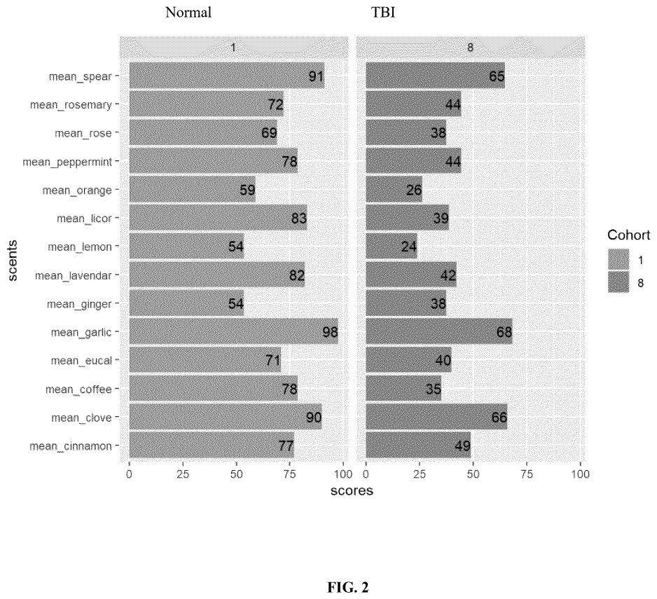

In the case of traumatic brain injury, AROMA™ testing on a limited cohort of subjects with confirmed traumatic brain injury and a “normal” cohort not having traumatic brain injury resulted in the scores shown in FIG. 2. After applying a Bonferroni correction for multiple comparisons, with an adjusted significance level of approximately 0.0033, significant differences between the normal and TBI cohorts were observed for licorice, garlic, lavender and coffee (p<0.0033). Other scent scores including rose, spearmint, orange, cinnamon, clove, peppermint, eucalyptus and lemon showed significant differences at the 0.05 level, but not after correction for multiple comparisons (p<0.05). No significant difference was observed for the rosemary and ginger scent score (p>0.05).

Based on these results, a diagnostic method and test kit for detecting, screening and/or monitoring traumatic brain injury comprises a pathology-specific panel of fragrances at various concentrations including licorice, garlic, lavender and coffee (those scents showing a significant difference in the TBI cohort from normal) and including rosemary and/or ginger as control scents (those scents showing no significant difference in the TBI cohort from normal). The diagnostic fragrances used in the test kits may be natural or commercially available essential oils or may be formulated “synthetic” fragrance compositions as hereafter described having the selected fragrances.

### Alzheimer's Testing and Kit

In a study reported in the Journal of Alzheimer's Disease 81 (2021) 641-650, AROMA™ testing and subsequent analysis using a random forest architecture determined the relative influence of variables to differentiate normal from Alzheimer's disease/mild cognitive impairment:

| | Concen- | | Relative |
|---|---|---|---|
| Variable | tration | Value | influence |
| Lavender | 1X | Correct | 31.69 |
| Gender | — | Female | 18.59 |
| Lemon | 4X | Correct | 12.39 |
| Age | — | — | 12.25 |
| Clove | 1X | Correct | 8.17 |
| SST-Lemon | — | No | 2.94 |
| Garlic | 1X | Correct | 1.91 |
| Thick nasal drainage | — | No problem | 1.65 |
| Psychological domain of | — | 0 (no symptoms) | 1.52 |
| SNOT-22 | | | |
| Lavender | 2X | Correct | 1.44 |
| Detection of negative control | — | Detected | 1.41 |
| Lavender | 8X | Correct | 1.23 |
| Blow nose | — | No problem | 1.10 |

Based on these results, the inventors propose a diagnostic method and testing kit for detecting, screening and/or monitoring the progression of Alzheimer's disease using a pathology-specific panel of fragrances including lavender, lemon, clove and garlic at the concentrations listed above. The diagnostic fragrances used in the test kits may be natural or commercially available essential oils or may be formulated “synthetic” fragrance compositions as hereafter described having the selected fragrances.

### SCENT™ Kit and Compositions

Another aspect of the present invention is directed to an olfactory diagnostic device that the inventors have named SCENT™ (Synthetic Component Essential Oils Neurocognitive Trial) proposed for use in the detection, screening and/or monitoring of a specific disease state. An exemplary kit includes “synthetic oils” (synthesized diagnostic compositions of complex odors identifiable as essential oil fragrances or as other known fragrances) with high batch-to-batch reproducibility, as opposed to the essential oil compositions found in nature or otherwise commercially available. While the acronym references “essential oils” it should be understood that the synthetic oils” may include any composition of complex odors that can be recognized by the subjects as a known odor or fragrance.

In one exemplary embodiment, a diagnostic test SCENT™_TBI is proposed as a novel non-invasive diagnostic for the early detection of non-penetrating traumatic brain injury (TBI). The test is an affordable, accessible, essential oil fragrance-based test of OD appropriate for community-based settings which targets an identified critical need (non-invasive, accessible, remote monitoring appropriate biomarker).

The selected scents or fragrances for the SCENT™-TBI panel are associated with TBI and based on essential oil fragrances. This panel of scents will be correlated with clinical diagnosis and TBI severity to derive olfactory phenotypes that are diagnostic of TBI via traditional statistical analyses and machine learning (ML) as discussed above. The inventors propose that the use of olfactory and subject data in ML algorithms can both accurately classify disease state and predict earlier when TBI occurs thus allowing for earlier comprehensive care needs. The inventors utilize novel methodology in which complex odorants are used as olfactory stimuli, which the inventors hypothesize is reflective of head injury severity, clinical diagnosis, and reflective of functionally relevant olfaction. The SCENT-TBI™ diagnostic device is a noninvasive test that uses 4 to 6 essential oil fragrances at multiple concentrations and is designed for point-of-care use.

In another aspect, the invention is directed to a method of making diagnostic compositions having a specific fragrance such as an essential oil fragrance and diagnostic kits including such diagnostic compositions. In one embodiment, the new test kit SCENT-TBI™ test kit (Synthetic Component Essential Oils Neurocognitive Trial) includes a panel of 4 to 6 “synthetic oils” (4 to 6 synthesized chemical compositions, each correlating to an essential oil fragrance) which are highly batch to batch reproducible, have quantifiable shelf-lifes and which use primary components of the naturally occurring/commercially available essential oils with the highest correlation to TBI while not being predictive of other disease states. It is believed that the use of these novel, synthetic oils derived from complex essential oils in an objective test of olfaction will better reflect “real world” olfaction and neurologic pathology that is appropriate for community-based and primary-care use.

### Exemplary Process for Development of “Synthetic Oil” Panel

A pathology-specific panel of diagnostic fragrances may be developed for use in the detection, screening and/or monitoring of a specific disease state or other condition by first selecting the fragrances to be used in the panel, determining the blend composition of the diagnostic fragrances, producing and testing trial samples of the diagnostic blends, and developing and producing final compositions with appropriate packaging. An exemplary process for this development effort is outlined below:
Step 1—Based on results of the olfactory phenotype testing (e.g. AROMA™ studies), select 6 essential oils, 4 which are differentiating for the specific disease state or other condition and 2 which are not (controls).
Step 2—Establish validated analytical methods to characterize the primary components (preferably 3-5 odorants or components) and their respective concentrations. The “diagnostic blends” which will be referred to as Synthetic Component Essential Oils Neurocognitive Test (SCENT™), each composed preferably of <5 primary components. Establish the specification ranges of the components for the diagnostic blends.
Step 3—Prepare trial batches of all 6 diagnostic blends using independently produced or obtained, discrete components, with their blending into appropriate carrier oils. Conduct testing of the trial blends and make needed adjustments to the blend compositions based on the testing.
Step 4—Manufacture diagnostic blends and establish their shelf-stability, packaging, and presentation, enabling the appropriate blinding of samples for the clinical studies.

Odorants can be, and often are, a complex mixture of specific chemical entities. The relative ratios of these discrete chemical entities, and their absolute concentration in an essential oil, will influence how they are perceived as odors. It is necessary to have the analytical chemistry methodology in place to characterize both naturally-occurring oils and the intentionally-blended oils, SCENT™ (which will be generated with discrete chemical entities). Not only are these analytical methods critical to deconvolute the natural oils and identify the primary components to be used in the “diagnostic blends”, they are also essential to controlling the quality, shelf-stability and product specifications necessary to ensure batch-to-batch reproducibility of the “diagnostic blends” and the expiry dating for the blends. Lastly, it becomes critical to provide the appropriate packaging and patient presentation of these diagnostic blends, which provides appropriate shelf-life and ability to blind supplies for clinical studies.

For Step 2 above, validated analytical methods adapted from the literature may be developed using HPLC, GC-MS, LCMS-MS methods. External calibration curves using synthetically derived and purchased components may be established, especially for the quantitation necessary to establish the specification ranges of the components. These methods may be used to examine the composition of the 6 different naturally-occurring essential oils identified from the AROMA™ studies, 4 demonstrating highly correlative and specific predictability for TBI and 2 essential oils which do not correlate (controls). The composition of the naturally-occurring essential oils can often contain more than 20-100 different discrete chemical entities. Analytical methods may be used to identify the primary components in each essential oil.

A “primary component” for purposes of this invention is defined as being one of the six most abundant species of odorants or components in the essential oil. If any of the 6 components are <5% of the total mixture, these components may not be considered for the final product. Characterization of the components may be performed employing multiple analytical methods. The calibration curves may be generated using pure single component reference standards, with regression analysis carried out. While it is anticipated that the total number of primary components to be used in the final formulation of the SCENT™ will be 5 or less, in some instances ranging from 2 to 5, in some instances ranging from 2 to 3, it should be understood that the relative amount of the components could be modified based on testing results. Rather than limiting any specific SCENT™ diagnostic composition to <5 components, expanding the component numbers to >5 may be explored for specific fragrances or panels.

Based on the results from characterization of components using multiple analytical chemistry methods, a single validated method may be selected for product release. The analytical information may be coupled with available literature information on the specific oils, recognizing that the composition of any specific essential oil is highly dependent on the supplier, the batch and the extraction method used to obtain it. From this combined information, the desired composition and concentrations of the 6 diagnostic blends may be determined. Specifically, knowledge from the literature of the typical primary components for various essential oils may be used to identify reference standards of specific chemical entities which should be purchased and used to quantitate the primary components of the essential oils that were in the AROMA™ studies. Preferably the <5 highest level components will be selected and quantified relative to one another. External calibration curves may be generated and used to determine the relative amounts of those primary components relative to one another in the designated essential oils.

For Step 3, the discrete primary chemical components of the essential oils identified in Step 2 may be purchased as a reference standard and verified by the analytical methods adopted in Step 2. If there is a primary component that cannot be obtained as a reference standard, the compound may alternatively be isolated, purified and identified, after which it may be synthesized, characterized, and released as an analytical reference standard. Samples of the 6 diagnostic blends, SCENT™, may be prepared at specific composition ratios, preferably from less than 5 constituents as determined in Step 2. Several carrier oils (e.g., fractionated coconut, mineral, jojoba) may be selected to evaluate as the base for the formulations. The discrete components for all 6 essential oils may be purchased and added to these carrier oils in the appropriate compositions and concentrations. Diagnostic blends (defined ratios of components) may be prepared with the sum of concentrations of the constituents, in some embodiments ranging from about 0.1-20% total concentration in the carrier oil, in some embodiments ranging from about 0.1-10% total concentration in the carrier oil, and in some embodiments ranging from about 8-12% total concentration in the carrier oil. It is anticipated that the blends for most fragrances can be made as about a 10% concentration (sum total of all components is about 10%) in at least one of the carrier oils. If not, other carrier oils may be considered or the target concentration may be lowered such as to about 5%.

The chemical stability of the diagnostic blends may be studied at 4° C., 25° C. and 40° C. for up to 9 months in sealed glass vials. Stability as function of the carrier oil and as a function of total concentration (0.1-10%) within the carrier oils may be monitored by using the analytical methods from Step 2. A targeted level of decomposition is preferably less than 10% change in the amount of any component over a 9 month study (a critical specification may be set at <15%). It is expected that the stability of the mixed components in the carrier oil will result in less than 10% degradation of any specific component over 9 months at 25° C. If not, backward iteration and analysis of the potential interactions between components may be conducted in an effort to determine the cause of instability. The inclusion of antioxidants in the formulation may also be considered, if necessary or desirable to achieve the preferred stability.

In Step 4, the diagnostic blends, SCENT-TBI™, may be manufactured in the selected carrier oil under appropriate conditions at the specific composition ratios as determined in Steps 2 and 3. Diagnostic blends (defined ratios of components) will be prepared with the sum of concentrations of the constituents preferably ending up at 0.1-10% of the concentration of the selected carrier oil. Stability of the selected final formulations may also be verified in the package(s) of choice. It is not anticipated that there will be no stability issues in clinical-destined packaging. However, if any stability issues are observed, the issues should be addressed with modified packaging, or if necessary, by modifying the formulation for packaging that is appropriate. SCENT™ kits using the diagnostic blends may be used in testing trials comparing the diagnostic blends (e.g. “synthetic oils”) to natural essential oils packaged in neutral “blinded” packaging.

### Examples of “Synthetic Oil” Formulation Development

### Lavender

A formulation for a lavender synthetic composition was developed by reviewing the literature to ascertain reported odorants contained within lavender essential oils, and conducting UPLC and GC analysis on commercially available lavender essential oils. The results of such review and analysis are listed below:

### Literature Review

a. Linalyl acetate
b. Linalool
c. 1,8-cineole
d. Terpinene-4-ol
e. Camphor
f. Lavandulyl acetate

### Peaks Seen by UPLC

a. Linalyl acetate
b. Linalool
c. 1,8-cineole
d. Terpinene-4-ol
e. Lavandulyl acetate-lack material
f. beta-caryophyllene
g. 4 unknown

### Peaks Seen by GC

a. 1,8-cineole
b. Terpinene-4-ol
c. 2 unknown

A formulation for a base composition consisting of odorants was developed based on the UPLC/GC data and literature review as follows:

| Components | Amount | |
|---|---|---|
| Linalyl acetate | 13 | μl |
| Linalool | 15 | μl |
| 1,8-cineole | 0.3 | μl |
| Terpinene-4-ol | 6.5 | μl |
| Beta-caryophyllene | 3.5 | μl |

### Clove

A formulation for a clove synthetic composition was developed by reviewing the literature to ascertain reported odorants contained within lavender essential oils, and conducting UPLC and GC analysis on commercially available lavender essential oils. The results of such review and analysis are listed below:

### Literature

a. Eugenol
b. Eugenol acetate
c. Beta caryophyllene

### Peaks Seen by UPLC

a. Eugenol
b. Eugenol acetate
c. 4 unknown
d. From manufacturer data we see 3 peaks and all identified (+beta-caryophyllene)

### Peaks Seen by GC

a. Eugenol
b. Eugenol acetate

A formulation for a base composition consisting of odorants was developed based on the UPLC/GC data and literature review as follows:

| Components | Amount | |
|---|---|---|
| Eugenol | 18 | μl |
| Eugenol acetate | 6.2 | μl |
| Beta-caryophyllene | 2.0 | μl |

### Spearmint

A formulation for a spearmint synthetic composition was developed by reviewing the literature to ascertain reported odorants contained within lavender essential oils, and conducting UPLC and GC analysis on commercially available lavender essential oils. The results of such review and analysis are listed below:

### Literature Review

a. Carvone
b. 1,8-cineole
c. Menthol
d. Menthone
e. Limonene

### Peaks Seen by UPLC

a. Carvone
b. 1,8-cineole (can only see secondary peaks, not main)
c. Menthol
d. Menthone (similar RT to cineole)
e. Limonene
f. 2 unknown

### Peaks Seen by GC

a. Carvone
b. Diallyl sulfide
c. 2 unknown

A formulation for a base composition consisting of odorants was developed based on the UPLC data and literature review as follows:

| Components | Amount | |
|---|---|---|
| Carvone | 26 | μl |
| Limonene | 2.3 | μl |
| 1,8-cineole | 3.2 | μl |

### Lemon

A formulation for a lemon synthetic composition was developed by reviewing the literature to ascertain reported odorants contained within lavender essential oils, and conducting UPLC/GC and GC analysis on commercially available lavender essential oils. The results of such review and analysis are listed below:

### Literature Review

a. Limonene
b. Beta-pinene
c. Gamma-terpinene
d. Alpha-pinene

### Peaks Seen by UPLC

a. 3 components have 3 RT via UPLC
b. 2 unknown in UPLC
c. Limonene
d. Beta-pinene
e. Gamma-terpinene
f. Alpha-pinene

### Peaks Seen by GC

a. Beta-pinene
b. 3 unknown

A formulation for a base composition consisting of odorants was developed based on the UPLC data and literature review as follows:

| Components | Amount | |
|---|---|---|
| Limonene | 27 | μl |
| Beta-pinene | 5.0 | μl |
| Gamma-terpinene | 2.0 | μl |
| Alpha-pinene | 2.0 | μl |

### Peppermint

A formulation for a peppermint synthetic composition was developed by reviewing the literature to ascertain reported odorants contained within lavender essential oils, and conducting UPLC/GC and GC analysis on commercially available lavender essential oils. The results of such review and analysis are listed below:

### Literature Review

a. L-menthol
b. L-menthone
c. Menthyl acetate
d. 1,8-cineole
e. Limonene
f. Carvone

### Peaks Seen by UPLC

a. L-menthol
b. L-menthone
c. 1,8-cineole
d. Limonene
e. 5 unknown
f. 2 unidentifiable from literature
i. Menthyl acetate
ii. Carvone

### Peaks Seen by GC

a. L-menthol
b. L-menthone
c. 3 unknown
d. 4 unidentifiable from literature (same as UPLC)

A formulation for a base composition consisting of odorants was developed based on the UPLC/GC data and literature review as follows:

| Components | Amount | |
|---|---|---|
| Menthol | 21 | μl |
| Menthone | 11 | μl |
| 1,8-cineole (potentially eliminate) | 2.3 | μl |
| Limonene | 8.0 | μl |
| Menthyl acetate | 2.0 | μl |

### Rosemary

A formulation for a rosemary synthetic composition was developed by reviewing the literature to ascertain reported odorants contained within lavender essential oils, and conducting UPLC and GC analysis on commercially available lavender essential oils. The results of such review and analysis are listed below:

### Literature Review

a. 1,8-cineole
b. Alpha-pinene
c. Camphor
d. Beta-pinene
e. Camphene
f. Beta-caryophyllene

### Peaks Seen by UPLC

a. 1,8-cineole
b. Camphor
c. 2 unknown
d. 4 unidentifiable

### Peaks Seen by GC

a. Camphor
b. 3 unknown
c. 5 unidentifiable from literature

A formulation for a base composition consisting of odorants was developed based on the UPLC/GC data and literature review as follows:

| Components | Amount | |
|---|---|---|
| 1,8-cineole | 21 | μl |
| Alpha-pinene | 8.0 | μl |
| Camphor | 7.0 | μl |
| Beta-pinene | 2.0 | μl |
| Camphene | 4.0 | μl |
| Beta-caryophyllene | 2.0 | μl |

### Eucalyptus

A formulation for a eucalyptus synthetic composition was developed by reviewing the literature to ascertain reported odorants contained within lavender essential oils, and conducting UPLC and GC analysis on commercially available lavender essential oils. The results of such review and analysis are listed below:

### Literature Review

a. 1,8-cineole
b. Limonene
c. Para-cymene
d. Alpha-pinene

### Peaks Seen by UPLC

a. Limonene
b. 1,8-cineole
c. 5 unknown
d. 3 unidentifiable from literature

### Peaks Seen by GC

a. Limonene
b. 3 unknown
c. 4 unidentifiable from literature

A formulation for a base composition consisting of odorants was developed based on the UPLC/GC data and literature review as follows:

| Components | Amount | |
|---|---|---|
| 1,8-cineole | 54 | μl |
| Limonene | 5.0 | μl |
| Para-cymene | 3.0 | μl |
| Alpha-pinene | 2.0 | μl |

### Cinnamon

A formulation for a cinnamon synthetic composition was developed by reviewing the literature to ascertain reported odorants contained within lavender essential oils, and conducting UPLC and GC analysis on commercially available lavender essential oils. The results of such review and analysis are listed below:

### Literature Review

a. Eugenol
b. Beta-caryophyllene
c. Benzyl benzoate
d. Linalool
e. Eugenol acetate

### Peaks Seen by UPLC

a. 5 unknown
b. 5 unidentifiable from literature

### Peaks Seen by GC

a. Eugenol

A formulation for a base composition consisting of odorants was developed based on the UPLC/GC data and literature review as follows:

| Components | Amount | |
|---|---|---|
| Eugenol | 80 | μl |
| Beta-caryophyllene | 2.0 | μl |
| Benzyl benzoate | 3.0 | μl |
| Linalool | 15 | μl |
| Eugenol acetate | 2.0 | μl |

### Licorice

A formulation for a licorice synthetic composition was developed by reviewing the literature to ascertain reported odorants contained within lavender essential oils, and conducting UPLC and GC analysis on commercially available lavender essential oils. The results of such review and analysis are listed below:

### Literature Review

a. Trans-anethole
b. Limonene
c. Alpha-pinene
d. Methyl chavicol (4-Allylanisole)

### Peaks Seen by UPLC

a. 4 unknown
b. 6 unidentifiable from literature

### Peaks Seen by GC

a. 3 unknown
b. 6 unidentifiable from literature

A formulation for a base composition consisting of odorants was developed based on the UPLC/GC data and literature review as follows:

| Components | Amount | |
|---|---|---|
| Trans-anethole | 72 | μl |
| Limonene | 10 | μl |
| Alpha-pinene | 3.0 | μl |
| Methyl chavicol | 2.0 | μl |

### Study Comparing Synthetic Oil Blends to Natural Essential Oils

A cross-over clinical trial design to confirm that the new synthetic controlled oils match earlier studies using natural essential oils as a method for diagnosis or progression of a specific disease state may be conducted. An exemplary study conducted relative to a pathology-specific panel of fragrances developed for TBI may include the following steps:
Step A: Determine strength of correlations between AROMA™ and SCENT™ panels and primary outcome measures (see Table 1—Experimental Plan below).
Step B: Analyze data using ML algorithms and determine differences in performance characteristics (sensitivity, specificity, negative predictive value, positive predictive value, overall accuracy) of algorithms that consider AROMA v. SCENT variables for the classification of TBI severity status, clinical outcomes, and neurocognitive performance.
Step C: Determine if subjects can differentiate between AROMA and SCENT odorants.

The extensive degree of anatomic and functional overlap between olfactory and neurocognitive processing provides a neuroanatomic basis for the OD seen in neurocognitive disorders such as TBJ. The brain recognizes odorants as “odor objects,” a process that is highly dependent on odorant concentration. For example, complex odors are recognized as discrete entities from their major chemical component. As such, synthetic creation of odorants for use in clinically relevant olfactory testing requires that these odorants perform equivalently to their essential oil, whole odorant, counterparts and contain the major components of the essential oils.

For this study, olfactory and clinical data including TBI severity, neurocognitive performance, and disease-specific and overall quality of life assessments may be collected. The performance on AROMA™ and SCENT™ may be correlated with primary outcome measure. Machine learning algorithms may then be tasked to correctly classify subjects into clinical category (mild, moderate, or severe TBI) using AROMA™ or SCENT™ variables. The inventors hypothesize that the algorithms will perform similarly and that correlations will not be significantly different. The odorants will be presented in pairs and it will be determined if subjects can perceive when the source of the paired odorants (AROMA™ and/or SCENT™) is different. The inventors anticipate that SCENT™ will be non-inferior to AROMA™ for prediction of biomarker and clinical status. It is also hypothesized that subjects will not be able to distinguish between AROMA™ and SCENT® odorants.

TABLE 1
Experimental Plan

| | | | |
|---|---|---|---|
| Test (* indicates | | | |
| primary outcome | | | |
| metric) | Domain Tested | Brief Description | Output metrics |

BrainCheck Tablet-based neurocognitive assessment

| Cognitive Quotient | Combination of the | The CQ score is a combination of the cognitive | |
|---|---|---|---|
| (CQ)* | below six tests | assessments and normalized to 100 being the | |
| | | mean, 15-point standard deviation, total range in | |
| | | 0-200. The normalized scores follow a standard | |
| | | normal distribution (0 as mean and 1 as standard | |
| | | deviation), where a lower score indicates lower | |
| | | performance and cognitive functioning. Subjects | |
| | | are categorized as low (>1 SD below mean), | |
| | | average, or high (>1 SD above mean) performers | |
| Trail Making A | Visual | Trace trail based on | Time to completion |
| | attention | ascending numbers | Number of Errors |
| | Task | | Percentile performance |
| | switching | | |
| Trail Making B | Visual | Trace trail of alternating | Time to completion |
| | attention | ascending numbers and | Number of errors |
| | Task | letters | Percentile performance |
| | switching | | |
| Digit Symbol | General | Match an arbitrary | Number matches made |
| Substitution Test | cognitive | correspondence of | Percentile performance |
| | performance | symbols to digits; when | |
| | | presented with a new | |
| | | symbol, they find as | |
| | | quickly as possible the | |
| | | corresponding digit and | |
| | | answer by pressing the | |
| | | digit. This is a | |
| | | continuous performance | |
| | | task in which the | |
| | | participant makes as | |
| | | many correct matches as | |
| | | possible within a fixed | |
| | | testing period. | |
| Stroop Test | Time to | Name of a color is | Number of colors |
| | overcome | printed in an | correctly and incorrectly |
| | cognitive | incongruent color and | selected during time |
| | interference | participant must select | period |
| | Executive | the correct color | Percentile performance |
| | function | | |
| Immediate and | Immediate | 10 words are displayed | Number of correct and |
| Delayed Recall Tests | recall | and the participant is | incorrect selections |
| | Delayed | then presented with | Percentile Performance |
| | recall | words and asked if the | |
| | | word is one of the | |
| | | previously displayed | |
| | | words or a distractor | |
| Flanker Task | Attention | Participants are | Number of correct and |
| | | presented with a target | incorrect selections |
| | | item flanked by | Percentile Performance |
| | | congruent or | |
| | | incongruent items. | |
| | | Participants identify the | |
| | | direction of the target as | |
| | | quickly and accurately | |
| | | as possible | |

AROMA and SCENT-TBI ™ Olfactory Assessment

| | | | |
|---|---|---|---|
| Scent-specific | 14-scents are assessed | Concentration-specific | 4 different concentrations |
| performance | with subjects | performance | are utilized to assess for |
| | indicating if they | | subtle deficits as well as |
| | detect a scent and | | true olfactory reserve. |
| | then selecting from a | | |
| | 4-option multiple | | |
| | choice test as to the | | |
| | scent detected. | | |
| | For SCENT-TBI, | | |
| | only 4 scents are | | |
| | used. | | |
| | | Composite Score* | For each scent, |
| | | | participants receive a |
| | | | score of 0, 25, 50, 75, or |
| | | | 100, corresponding to |
| | | | never responding |
| | | | correctly, or correct |
| | | | responses to the 8X, 4X, |
| | | | 2X, and 1X, respectively. |
| | | | An overall composite |
| | | | score can be calculated |
| | | | by averaging the scent- |
| | | | specific scores. |

Sinonasal Outcome Test-22 (SNOT-22)*

| 22 question inventory | Symptom-based | Higher score (maximum of 110) indicates higher |
|---|---|---|
| about sinonasal | rhinosinusitis | symptom burden. |
| symptoms and health | outcome measure | |
| | scored from 0 to 5 | |
| | regarding rhinologic, | |
| | extranasal, ear/facial, | |
| | psychologic, and | |
| | sleep symptoms. | |

Neurologic Symptom Inventory (NSI)*

| 22-item self-report | Likert scale from 0-4 | Total scores range from 0 to 88, with higher |
|---|---|---|
| questionnaire of | used to report the | scores indicating more severe symptom burden |
| sinonasal health | presence and severity | |
| assessing the severity | of common | |
| of somatic, cognitive, | concussion | |
| and affective | symptoms. | |
| symptoms over the | | |
| prior two weeks. | | |

Mild Brain Injury Atypical Symptom Scale (mBIAS)

| 8-item questionnaire of | Likert scales of 0-4 | Total symptom score range of 0-32 with higher |
|---|---|---|
| rationally derived items | (none to very severe | score indicating more symptoms. Scores ≥7 |
| uncommonly endorsed | symptoms) | indicate possible symptom exaggeration. |
| following concussion. | | |
| Questions are | | |
| interspersed with the | | |
| NSI. | | |

SF-8*

| 8 question inventory | Subjective rating of | Norm-based scoring is used with score of 50 |
|---|---|---|
| assessing general | overall health | being what is expected in the general population. |
| health, physical | (excellent to very | Score differences of 10 points equates to 1 SD. |
| functioning, physical | poor), impact of | |
| role bodily pain, | physical health | |
| vitality, social | problems on activities | |
| functioning, mental | (“none at all” to | |
| health, and emotional | “could not do”), and | |
| role. | emotional and pain | |
| | experience. | |

Questionnaire of olfactory disorders-negative statements (QoD-NS)*

| 17 negative statements | Statements about the | Higher scores (maximum score of 71) indicate |
|---|---|---|
| regarding olfaction and | impact of olfaction on | greater negative impact on quality of life. |
| perception of olfactory | different olfaction- | |
| deficits | specific negative | |
| | statements are | |
| | provided and subjects | |
| | are asked to select | |
| | how much they agree | |
| | with the statement | |
| | (“totally disagree” to | |
| | “totally agree). | |

### Study Procedure

Subjects age 18+ who are healthy controls as well as those who have experienced mild, moderate, or severe TBI in the past 6 months will be recruited. Collected demographic information includes age, gender, race/ethnicity, and education level as theses variables are all known confounders of olfactory performance.

After collection of demographic and clinical information, subjects will complete olfactory testing with AROMA™ per prior protocol discussed above. Briefly, they will be presented with 14 scents at multiple concentrations (1×, 2×, 4×, and 8×). While all subjects begin at the same standardized concentration, presentation of additional concentrations of a particular scent depends on performance. For example, if a subject answers incorrectly, they are presented with the next higher concentration. Conversely, correct response prompts a lower concentration in the following round. Following AROMA™, subjects will experience an “olfactory break,” during which time they will undergo plasma biomarker testing via blood draw. They will then complete SCENT™, in which the synthetic components identified and synthesized as described above are dilute into analogous concentrations and presented to subjects for olfactory testing. Following SCENT™, a second olfactory break will concur, during which time subjects will complete the Sinonasal Outcomes Test (SNOT-22), which ensures that olfactory performance is not compounded by sinonasal disease, as well as the Questionnaire of Olfactory Disorders-Negative Statements to determine impact of OD on QoL. Subjects will then undergo their final olfactory task, the goal of which is to determine if subjects can differentiate between AROMA™ and SCENT™ odorants. They will be presented with scents from AROMA™ and SCENT™ and asked to determine if the olfactory stimuli are the same or different. Four concentrations of four scents each from AROMA™ and SCENT™ will be presented, for a total of 16 paired tests. In 8 of the tests, the odorants will be the same (e.g. both from AROMA™ or both from SCENT™). In the remaining 8 tests, one odorant will be from AROMA™ and the other from SCENT™. The odorant and concentration used for these paired tests will be randomly selected and assigned via computer algorithm on the day of testing (see Table 2 below).

TABLE 2

| Common | | | | Correct |
|---|---|---|---|---|
| Name | Concentration | Odorant 1 | Odorant 2 | response |
| Lavender | 2X | AROMA ™ | SCENT ™ | Different |
| Lemon | 4X | AROMA ™ | AROMA ™ | Same |
| Rose | 8X | SCENT ™ | SCENT ™ | Same |

Data may be analyzed with SPSS version 26 (Armonk, NY) and R. Differences among diagnostic groups may be assessed using 2 for categorical variables and one-way ANOVA tests for scale variables. When significant differences between groups are noted, Tukey's post-hoc analysis may be utilized. If parametric assumptions are not met, Kruskal-Wallis tests and Dunn's post-hoc tests may be used. Univariate correlations between scale variables may be assessed using Spearman's rank test. To further explore the role of covariates on the olfactory and biomarker endpoints, a generalized linear mixed model for repeated measures may be developed. Gamma regression may be applied (gamma distribution with a log link) and any subject with an AROMA™/SCENT™ score of 0 will have their scores imputed as half the lowest possible score (1%). The model may be fitted via maximum likelihood and consider the following fixed effects: age, gender, and education; additionally, it may treat each subject as a random effect.

Interaction with time may be allowed for all covariates. Non-significant variables may be hierarchically removed one by one starting with the highest p-value, until only the variables with at least trend significance remain in the model (p<0.1). Machine learning architectures may be evaluated for their ability to correctly classify (1) TBI status (mild, moderate, or severe TBI) based on olfactory data. Primary variables of interest include composite and scent-specific AROMA™/SCENT™ scores. This data may be interrogated to determine variables—individually or in combination—with the strongest relative influence in predicting cognitive status. Multiple machine learning models may be compared to determine best fit, random forest, gradient boost, Xgboosting, LASSO, and artificial neural networks, which have been shown to have excellent performance in making clinical predictions with health data. Models may be trained using the test data. 4-fold cross validation and hyper parameters may be tuned to identify the candidate model with the highest predictive accuracy and, secondarily, negative predictive value. “Majority-voting” may be investigated to integrate different machine/deep learning algorithms to optimize the prediction performance. Models may be developed and AUC based on ROC analysis reported.

The final model may be determined based on measure of fit. Given a long-term goal of home- or primary-care based use of OD testing, the sensitivity, specificity, and predictive value of olfactory phenotype derived algorithms should meet or exceed those of other available screening tests that are administered in a point-of-care setting with real-time results. For example, prior studies have shown single clinical measures such as the Montreal Cognitive Assessment to have sensitivity ranging from 55% to 75% and specificity between 52.5% and 100%. These parameters may be used as the benchmark of the algorithms and require sensitivity of >70% and specificity>75%.

Traditional power analysis cannot be done for machine learning. Instead, a logistic model may be used to simulate training data with binary outcomes and five important risk factors along with 15 noise variables. It is believed that accurate prediction with a sample size of 100 (or 33 individuals per group) may be achieved in this simplified classification. To account for unbalanced allocations and high-dimensional predictors, it is desired to over-recruit by 20% and anticipate a total sample size of 120 or 40 subjects per group.

### Aroma/Nasal Inhalers

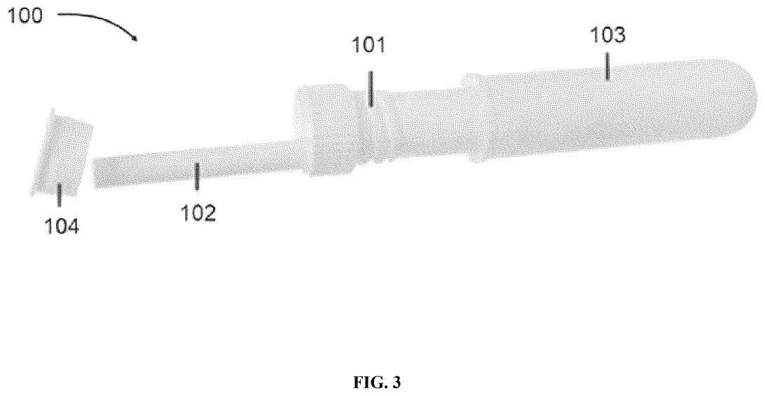

As shown in FIG. 3, a typical aroma or nasal inhaler 100 that can be used in the testing kits described herein comprises an absorbent material 102 and container 101, such as a cylinder, for containing the absorbent material 102 that contains perforations to allow inhalation of the aromas inside the container and a cap 104 for replacing the absorbent material 102. The absorbent material 102 can be dosed with a known amount of one or more diagnostic fragrances (e.g. essential oils or the synthetic oils described herein) and then inserted into the cylinder. The device also optionally includes a cover 103 to seal the aromas inside the device when not in use. Aromatherapy inhalers are commercially available and can be adapted for use in the disclosed devices, its, and methods.

### Answer Key

The answer key that may be used with the testing kits can take any suitable form, depending on the environment. In some cases, the answer key is a computer interface. The computer interface can be accessible using remote devices such as smartphones, tablets or other hand-held devices for easy use in the field. In some cases, the answer key is a document, chart, card, or book. In some cases, the answer key is designed to be filled out by a medical professional recording the subject's answers. In other cases, the answer key is designed to be followed and filled out by the subject. In these embodiments, the answer key can include an additional prompt to make sure the subject selects the correct inhaler.

### Computing Device

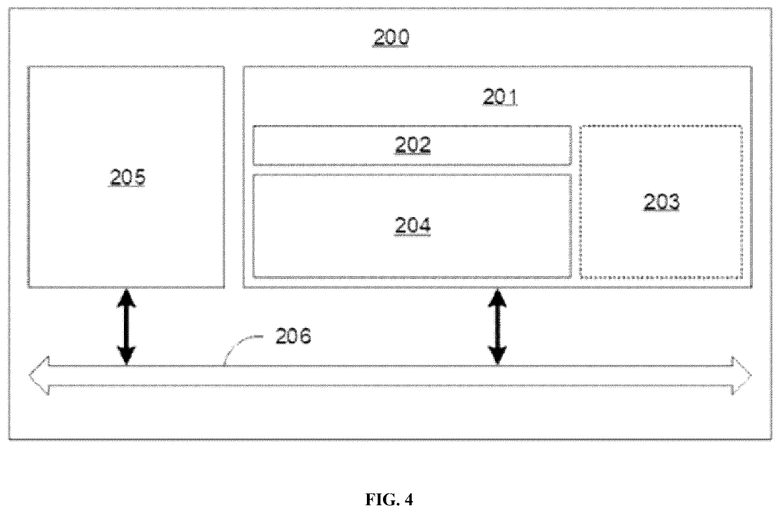

Referring now to FIG. 4, shown is an example of a computing device 200 that can be used in the methods and kits described herein. The computing device 200 can include at least one processor circuit, for example, having a processor 205 and a memory 201, both of which are coupled to a local interface 206. To this end, the computing device(s) 200 may comprise, for example, a computer, laptop, smartphone, tablet, or other mobile processing unit providing computing capability. The computing device(s) 200 may include, for example, one or more display devices such as cathode ray tubes (CRTs), liquid crystal display (LCD) screens, gas plasma-based flat panel displays, LCD projectors, or other types of display devices, etc. The computing device(s) 1103 may also include, for example various peripheral devices. In particular, the peripheral devices may include input devices such as, for example, a keyboard, keypad, touch pad, touch screen, microphone, scanner, mouse, joystick, or one or more push buttons, etc. Even though the computing device 200 is referred to in the singular, it is understood that a plurality of computing devices 200 may be employed in the various arrangements as described above. The local interface 206 may comprise, for example, a data bus with an accompanying address/control bus or other bus structure as can be appreciated.

Stored in the memory 201 are both data and several components that are executable by the processor 205. In particular for use with the testing kits, stored in the memory 201 and executable by the processor 205 are an answer key 206 and potentially other applications such as machine learning algorithms predictive for one or more disease states or other conditions. Also stored in the memory 201 may be a data store 203 and other data. In addition, an operating system 202 may be stored in the memory 201 and executable by the processor 205. The data store 203 may be may be located in a single computing device or may be dispersed among many different devices.

It is understood that there may be other applications that are stored in the memory 201 and are executable by the processor 205 as can be appreciated. Where any component discussed herein is implemented in the form of software, any one of a number of programming languages may be employed such as, for example, C, C++, C#, Objective C, Java, Java Script, Perl, PHP, Visual Basic, Python, Ruby, Delphi, Flash, or other programming languages.

A number of software components are stored in the memory 201 and are executable by the processor 205. In this respect, the term “executable” means a program file that is in a form that can ultimately be run by the processor 205. Examples of executable programs may be, for example, a compiled program that can be translated into machine code in a format that can be loaded into a random access portion of the memory 201 and run by the processor 205, source code that may be expressed in proper format such as object code that is capable of being loaded into a random access portion of the memory 201 and executed by the processor 205, or source code that may be interpreted by another executable program to generate instructions in a random access portion of the memory 201 to be executed by the processor 205, etc. An executable program may be stored in any portion or component of the memory 201 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 201 is defined herein as including both volatile and nonvolatile memory and data storage components. Volatile components are those that do not retain data values upon loss of power. Nonvolatile components are those that retain data upon a loss of power. Thus, the memory 201 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 205 may represent multiple processors 205 and the memory 201 may represent multiple memories 201 that operate in parallel processing circuits, respectively. In such a case, the local interface 206 may be an appropriate network that facilitates communication between any two of the multiple processors 205, between any processor 205 and any of the memories 201, or between any two of the memories 201, etc. The local interface 206 may comprise additional systems designed to coordinate this communication, including, for example, performing load balancing. The processor 205 may be of electrical or of some other available construction.

Although the answer key, and other various systems described herein, may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits having appropriate logic gates, or other components, etc. Such technologies are generally well known by those skilled in the art and, consequently, are not described in detail herein.

Also, any logic or application described herein, including an answer key that comprises software or code can be embodied in any non-transitory computer-readable medium for use by or in connection with an instruction execution system such as, for example, a processor in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. The computer-readable medium can comprise any one of many physical media such as, for example, electronic, magnetic, optical, electromagnetic, infrared, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

### Synthetic Fragrance Compositions

As discussed above, the diagnostic fragrances used for developing an olfactory phenotype and used in testing to detect, screen and/or monitor a specific disease state or other condition may be synthetic fragrance compositions formulated to be recognized as an essential oil fragrance or other known complex fragrance. In some embodiments, the synthetic fragrance composition comprises a limited set or number of odorants (e.g. single aromatic compounds or molecules) in prescribed amounts. In some embodiments, the synthetic fragrance composition comprises a “base composition” consisting of the limited set of odorants in prescribed amounts and a carrier, such as an oil. The concentration or dosage amounts of the synthetic fragrance composition can be varied by varying the amount of the base composition in the carrier. In some embodiments, the base composition consists of two to five different types of odorants, in some embodiments at least three different types of odorants. In some embodiments, the base composition may be provided in a carrier at a concentration ranging from about 0.1-99.9% total concentration in the carrier, in some embodiments ranging from about 0.1-20% total concentration in the carrier, in some embodiments ranging from about 0.1-10% total concentration in the carrier, and in some embodiments ranging from about 8-12% total concentration in the carrier. Suitable carriers include oils such as fractionated coconut, mineral, and jojoba oils, albeit other carriers known or later developed in the art may be used. In addition, the carriers may include preservatives such as anti-oxidants to assist in preserving and increasing the shelf-life of the composition for use.

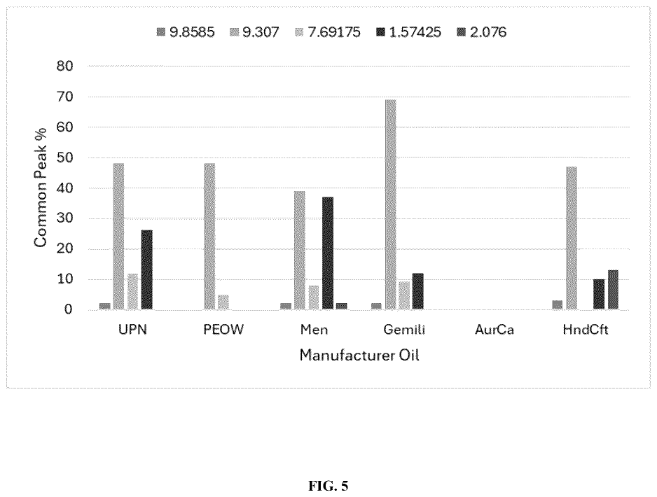

While the inventors have determined that complex fragrance compositions are more selective and provide better results for diagnostic testing than compositions containing only one odorant, they have also found that commercially available complex fragrances, such as essential oil fragrances, are not uniform in composition amongst different manufacturers or even amongst different batches from the same manufacturer. For example, with reference to FIG. 5, when seven different commercially available lavender essential oil compositions were analyzed via gas chromatography, it was found that the compositions varied significantly as to the specific odorants included within the compositions and the percentage amounts of those compounds. While certain components/peaks were present and consistent throughout all of the lavender oils (such as the peak at 9.3 min.), the percentage amounts are not consistent and the presence and amounts of other components are not consistent.

In addition, the shelf life of commercially available fragrance compositions may vary due to the varied composition and may not be easily ascertained for use in the diagnostic kits since the date of manufacture or storage protocols prior to purchase may not be known. For purposes of the diagnostic testing it is very important to ensure consistency in the formulation of the compositions and to ensure consistency in shelf life for purposes of use in the test kits. Using a synthetic fragrance composition as opposed to a natural essential oil ensures uniformity and consistency in the results of the testing.

A synthetic lavender fragrance composition in accordance with the present invention comprises odorants selected from the group consisting of linalyl acetate, linalool, 1,8-cineole, terpinene-4-ol, beta-caryophyllene, and combinations of the foregoing, each in amounts ranging from about 0.01-100% by volume of the base composition. For purposes of this application, the base composition is the composition consisting of the odorants included within the diagnostic fragrance composition and not including the carrier or additives. In one embodiment, the lavender base composition comprises linalyl acetate, linalool and terpinene-4-ol. In some embodiments, the lavender base composition comprises linalyl acetate in an amount ranging from about 30-35%, linalool in amounts ranging from about 35-42%, and terpinene-4-ol in amounts ranging from about 12-18% of the base composition by volume. In some embodiments, the lavender base composition additionally comprises 1,8-cineole in amounts ranging from about 0.1-1% y volume of the base composition. In some embodiments, the base composition additionally comprises beta-caryophyllene in amounts ranging from about 5-12% of the base composition by volume. In some embodiments, the base formulation comprises linalyl acetate in amounts from about 33-40%, linalool ranging in amounts ranging from about 39-40%, 1,8-cineole in amounts ranging from about 0.6-0.8 percent, terpinene-4-ol in amounts ranging from about 16-17% and beta-caryophyllene ranging from about 9-10% of the base composition by volume.

A synthetic clove fragrance composition in accordance with the present invention comprises odorants selected from the group consisting of eugenol, eugenol acetate, beta-caryophyllene, and combinations of the foregoing, each in amounts ranging from about 0.1-100% of the base composition by volume. In some embodiments, the clove base composition comprises eugenol and eugenol acetate. In some embodiments, the base composition additionally comprises beta-caryophyllene. In some embodiments, the base composition comprises eugenol in amounts ranging from about 65-72% by volume of the base composition. In some embodiments, the base composition comprises eugenol acetate in amounts ranging from about 20-25% by volume of the base composition. In some embodiments, the base composition comprises beta-caryophyllene in amounts ranging from about 5-10% by volume of the base composition. In some embodiments, the base composition comprises eugenol in an amount ranging from about 68-69% by volume of the base composition, eugenol acetate in amounts ranging from about 23-24% by volume of the base composition and beta-caryophyllene in amounts ranging from about 7-8% by volume of the base composition.

A synthetic spearmint fragrance composition in accordance with the present invention comprises odorants selected from the group consisting of carvone, limonene, 1,8-cineole, and combinations of the foregoing, each of which is provided in amounts ranging from 0.1-100% by volume of the base composition. In some embodiments, the base composition comprises carvone and 1,-8 cineole. In come embodiments, the base composition comprises carvone in amounts ranging from about 80-85% by volume of the base composition. In some embodiments, the base composition comprises limonene in amounts ranging from about 5-10% by volume of the base composition. In some embodiments, the base composition comprises 1,8-cineole in amounts ranging from about 8-12% by volume of the base composition by volume. In some embodiments, the base composition comprises carvone in an amount ranging from about 82-83%, limonene in amounts ranging from about 7-8%, and 1,8-cineole in amounts ranging from about 9-11% by volume of the base composition.

A synthetic lemon fragrance composition in accordance with the present invention comprises odorants selected from the group consisting of limonene, beta-pinene, gamma-terpinene, alpha-pinene, and combinations of the foregoing each in amounts ranging from about 0.1-100% by volume of the base composition. In some embodiments, the base composition comprises limonene and beta-pinene. In some embodiments, the base composition comprises limonene in amounts ranging from about 70-80% by volume of the base composition. In some embodiments, the base composition comprises beta-pinene in amounts ranging from about 10-15% by volume of the base composition. In some embodiments, the base composition comprises gamma-terpinene in amounts ranging from about 4-6% by volume of the base composition. In some embodiments, the base composition comprises alpha-pinene in amounts ranging from about 4-6% by volume of the base composition by volume. In some embodiments, the base composition comprises limonene in an amount ranging from about 74-76%, beta-pinene in amounts ranging from about 13-14% of the base composition, gamma-terpinene in amounts ranging from about 5-6% of the base composition and alpha-pinene in amounts ranging from about 5-6% of the base composition by volume.

A synthetic peppermint fragrance composition in accordance with the present invention comprises odorants selected from the group consisting of menthol, menthone, 1,8-cineole, limonene, menthol acetate, and combinations of the foregoing, each of which may be provided in amounts ranging from about 0.1-100% by volume of the base composition. In some embodiments, the base composition comprises menthol, menthone and limonene. In some embodiments, the base composition comprises menthol in amounts ranging from about 45-50% by volume of the base composition. In some embodiments, the base composition comprises menthone in amounts ranging from about 20-25% by volume of the base composition. In some embodiments, the base composition comprises 1,8-cineole in amounts ranging from about 3-7% by volume of the base composition. In some embodiments, the base composition comprises limonene in amounts ranging from about 15-20% by volume of the base composition. In some embodiments, the base composition comprises menthol acetate in amounts ranging from about 3-5% by volume of the base composition. In some embodiments, the base composition comprises menthol in an amount ranging from about 47 to 48%, menthone in amounts ranging from about 24 to 25%, 1,8-cineole in amounts ranging from about 5-6%, limonene in amounts ranging from about 17-19%, and menthol acetate in amounts ranging from about 4-5% by volume of the base composition.

A synthetic rosemary fragrance composition in accordance with the present invention comprises odorants selected from the group consisting of 1,8-cineole, alpha-pinene, camphor, beta-pinene, camphene, beta-caryophyllene, and combinations of the foregoing each of which is provided in amounts ranging from about 0.1-100% by volume of the base composition. In some embodiments, the base composition comprises 1,8-cineole, alpha-pinene and camphor. In some embodiments, the base composition comprises 1,8-cineole in amounts ranging from about 45-50% by volume of the base composition. In some embodiments, the base composition comprises alpha-pinene in amounts ranging from about 15-20% by volume of the base composition by volume. In some embodiments, the base composition comprises camphor in amounts ranging from about 12-17% by volume of the base composition. In some embodiments, the base composition comprises beta-pinene in amounts ranging from about 3-6% by volume of the base composition. In some embodiments, the base composition comprises camphene in amounts ranging from about 7-11% by volume of the base composition. In some embodiments, the base composition comprises beta-caryophyllene in amounts ranging from about 3-6% by volume of the base composition. In some embodiments, the base composition comprises 1,8-cineole in amounts ranging from about 47-48%, alpha-pinene in amounts ranging from about 18-19%, camphor in amounts ranging from about 15-16%, beta-pinene in amounts ranging from about 4-5%, camphene in amounts ranging from about 8-10%, and beta-caryophyllene in amounts ranging from about 4-5% by volume of the base composition.

A synthetic eucalyptus fragrance composition in accordance with the present invention comprises odorants selected from the group consisting of 1,8-cineole, limonene, para-cynene, alpha-pinene, and combinations of the foregoing, each of which is provided in amounts ranging from about 0.1-100% by volume of the base composition. In some embodiments, the base composition comprises 1,8-cineole and limonene. In some embodiments, the base composition comprises 1,8-senial in an amount ranging from about 80-86% by volume of the base composition. In some embodiments, the base composition comprises lemonine in amounts ranging from about 5-10% by volume of the base composition. In some embodiments the composition comprises para-cynine in amounts ranging from about 3-6% by volume of the base composition. In some embodiments, the base composition comprises alpha-pineen in amounts ranging from about 2-5% of the base composition by volume. In some embodiments, the base composition comprises 1,8-senial in amounts ranging from about 84-85%, lemonine in amounts ranging from about 7-8%, para-Cynine in amounts ranging from about 4-5%, and alpha-pineen in amounts ranging from about 3-4% by volume of the base composition.

A synthetic cinnamon fragrance composition in accordance with the present invention comprises odorants selected from the group consisting of eugenol, beta-caryophyllene, benzyl benzoate, linalool, eugenol acetate, and combinations of the foregoing, each provided in amounts ranging from about 0.1-100% by volume of the base composition. In some embodiments, the base composition comprises eugenol and linalool. In some embodiments, the base composition comprises eugenol in amounts ranging from about 75-80% by volume of the base composition. In some embodiments, the base composition comprises beta-caryophyllene in amounts ranging from about 0.5-2% by volume of the base composition. In some embodiments, the base composition comprises benzyl benzoate in amounts ranging from about 1-3% of the base composition by volume. In some embodiments, the base composition comprises linalool in amounts ranging from about 12-16% of the base composition by volume. In some embodiments, the base composition comprises eugenol acetate in amounts ranging from about 0.5-3% by volume of the base composition. In some embodiments, the base composition comprises eugenol in amounts ranging from about 78-79%, beta-caryophyllene in amounts ranging from about 1-2%, benzyl benzoate in amounts ranging from about 2-3%, linalool in amounts ranging from about 14-15% and eugenol acetate in amounts ranging from about 1-2% by volume of the base composition.

A synthetic licorice fragrance composition in accordance with the present invention comprises odorants selected from the group consisting of trans-anethole, limonene, alpha-pinene, methyl chavicol, and combinations of the foregoing, of which may be provided in amounts ranging from about 0.1-100% by volume of the base composition. In some embodiments, the base composition comprises trans-antethole and limonene. In some embodiments, the base composition comprises trans-anethole in amounts ranging from about 80-85% by volume of the base composition. In some embodiments, the base composition comprises limonene in amounts ranging from about 9-13% by volume of the base composition. In some embodiments, the base composition comprises alpha-pinene in amounts ranging from about 2-5% by volume of the base composition. In some embodiments, the base composition comprises methyl chavicol in amounts ranging from about 1-3% by volume of the base composition. In some embodiments, the base composition comprises trans-anethole in amounts ranging from about 82-83%, limonene in amounts ranging from about 11-12%, alpha-pinene in amounts ranging from about 3-4%, and methyl chavicol in amounts ranging from about 1-3% by volume of the base composition.

A method of making a diagnostic fragrance for olfactory testing comprises combining a limited set of odorants to produce a synthetic fragrance composition that is recognized as an essential oil fragrance or other known complex fragrance as described herein. The fragrance composition may be produced by combining the odorants in prescribed amounts.

A method of detecting, screening and/or monitoring a specific disease state or other condition comprises using one or more of the synthetic fragrance compositions described herein in a pathology-specific panel of fragrances as described herein.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

1. A method of detecting, screening and/or monitoring a specific disease state or other condition in a subject, said method comprising:
a. Developing an olfactory phenotype for a specific disease state or other condition;
b. Selecting a pathology-specific panel of fragrances comprising a plurality of fragrances based on the olfactory phenotype;
c. Testing the subject's ability to detect and identify the fragrances within the pathology-specific panel of fragrances;
d. Comparing the subject's ability to detect and identify the fragrances within the pathology-specific panel of fragrances to the olfactory phenotype.

2. The method of claim 1, wherein the olfactory phenotype is developed by conducting olfactory phenotype testing on at least two cohorts of subjects, wherein a first cohort of subjects is suffering from the specific disease state or other condition, and a second cohort of subjects is not suffering from the specific disease state or other condition, the olfactory phenotype testing comprising:
a. first prompting each subject to smell a fragrance from a phenotype testing panel of fragrances;
b. second prompting each subject to indicate if an aroma is detected and recording a response from the subject;
c. if the subject's response to the second prompting step is that an aroma is detected, third prompting the subject to identify the aroma from among a list of choices and recording a response from the subject;
d. if the subject's response to the second prompting step is that an aroma is not detected, optionally repeating the prompting steps with a higher dose of the fragrance until the subject's response to the second prompting step is that an aroma is detected or until a pre-determined maximum dose is used and the subject's response to the second prompting step is that an aroma is not detected;
e. repeating the prompting steps with each fragrance in the phenotype testing panel of fragrances;
f. comparing the recorded responses of the first cohort to the recorded responses of the second cohort to identify any unique patterns of responses by the first cohort.

3. The method of claim 1, wherein at least one of the fragrances within the phenotype testing panel of fragrances is a complex fragrance comprising more than one odorant.

4. The method of claim 3, wherein each of the fragrances within the phenotype testing panel of fragrances is a complex fragrance comprising more than one odorant.

5. The method of claim 3, wherein the complex fragrance is a synthetic composition comprising a limited set of odorants.

6. The method of claim 2, wherein the pathology-specific panel of fragrances comprises a grouping or set of fragrances for which the recorded responses of the first cohort as to one or more fragrances or concentrations of fragrances creates a unique pattern of responses that is different from the pattern created from the recorded responses of the second cohort to the same grouping or set of fragrances or concentrations.

7. The method of claim 1, wherein each of the fragrances within the pathology-specific panel of fragrances is a complex fragrance comprising more than one odorant.

8. The method of claim 7, wherein the complex fragrance is a synthetic composition comprising at least two and no more than ten different odorants combined together in a predefined amount.

9. The method of claim 7 for detecting, screening and/or monitoring AD wherein the pathology-specific panel of fragrances is selected from the group consisting of lavender, spearmint, licorice, garlic, coffee, lemon, clove, and combinations thereof.

10. The method of claim 7 for detecting, screening and/or monitoring AD wherein the pathology-specific panel of fragrances is selected from the group consisting of lavender, lemon, clove, garlic and combinations thereof.

11. The method of claim 7 for detecting, screening and/or monitoring traumatic brain injury wherein the pathology-specific panel of fragrances is selected from the group consisting of licorice, garlic, lavender, coffee and combinations thereof.

12. The method of claim 7 for detecting, screening and/or monitoring traumatic brain injury wherein the pathology-specific panel of fragrances comprises control fragrances selected from the group consisting of rosemary, ginger and combinations thereof.

13. An olfactory diagnostic kit comprising a pathology-specific panel of fragrances for detecting, screening and/or monitoring a specific disease state or other condition in a subject, wherein each of the fragrances within the pathology-specific panel is a synthetic fragrance composition that is produced by combining two or more different odorants together in prescribed amounts.

14. An olfactory diagnostic kit according to claim 13 for detecting, screening and/or monitoring AD comprising a pathology-specific panel of fragrances selected from the group consisting of lavender, spearmint, licorice, garlic, coffee, lemon, clove, and combinations thereof.

15. An olfactory diagnostic kit according to claim 13 for detecting, screening and/or monitoring AD comprising a pathology-specific panel of fragrances selected from the group consisting of lavender, lemon, clove, garlic and combinations thereof.

16. An olfactory diagnostic kit according to claim 13 for detecting, screening and/or monitoring traumatic brain injury comprises a pathology-specific panel of fragrances selected from the group consisting of licorice, garlic, lavender, coffee and combinations thereof.

17. An olfactory diagnostic kit according to claim 13 for detecting, screening and/or monitoring traumatic brain injury that comprises control fragrances selected from the group consisting of rosemary, ginger and combinations thereof.

18. An olfactory diagnostic kit according to claim 13, the kit additionally comprising a plurality of aroma inhalers, each dosed with a fragrance from the pathology-specific panel of fragrances and an answer key.

19. An olfactory diagnostic kit according to claim 13, comprising a synthetic lavender fragrance comprising odorants selected from the group consisting of linalyl acetate, linalool, 1,8-cineole, terpinene-4-ol, beta-caryophyllene, and combinations thereof.

20. An olfactory diagnostic kit according to claim 13, comprising a synthetic clove fragrance comprising odorants selected from the group consisting of eugenol, eugenol acetate, beta-caryophyllene, and combinations thereof.

21. An olfactory diagnostic kit according to claim 13, comprising a synthetic spearmint fragrance comprising odorants selected from the group consisting of carvone, limonene, 1,8-cineole, and combinations thereof.

22. An olfactory diagnostic kit according to claim 13, comprising a synthetic lemon fragrance comprising odorants selected from the group consisting of limonene, beta-pinene, gamma-terpinene, alpha-pinene, and combinations thereof.

23. An olfactory diagnostic kit according to claim 13, comprising a synthetic peppermint fragrance composition comprising odorants selected from the group consisting of menthol, menthone, 1,8-cineole, limonene, menthol acetate, and combinations thereof.

24. An olfactory diagnostic kit according to claim 13, comprising a synthetic rosemary fragrance comprising odorants selected from the group consisting of 1,8-cineole, alpha-pinene, camphor, beta-pinene, camphene, beta-caryophyllene, and combinations thereof.

25. An olfactory diagnostic kit according to claim 13, comprising a synthetic eucalyptus fragrance comprising odorants selected from the group consisting of 1,8-cineole, limonene, para-cynene, alpha-pinene, and combinations thereof.

26. An olfactory diagnostic kit according to claim 13, comprising a synthetic cinnamon fragrance comprising odorants selected from the group consisting of eugenol, beta-caryophyllene, benzyl benzoate, linalool, eugenol acetate, and combinations thereof.

27. An olfactory diagnostic kit according to claim 13, comprising a synthetic licorice fragrance comprising odorants selected from the group consisting of trans-anethole, limonene, alpha-pinene, methyl chavicol, and combinations thereof.
